(19) United States
(12) Patent Application Publication
(10) Pub. No.: US 2008/0187090 A1
(43) Pub. Date: Aug. 7, 2008

(54) Tomographic Method

(76) Inventors: Martti Kalke, Tuusula (FI); Samuli Siltanen, Helsinki (FI); Henri Setala, Littoinen (FI)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/909,015

(22) PCT Filed: Apr. 12, 2006
(86) PCT No.: PCT/FI2006/050150

§ 371 Date: Sep. 18, 2007

(30) Foreign Application Priority Data
Apr. 13, 2005 (FI) 20055168

(51) Int. Cl. A61B 6/00

(52) U.S. Cl. 378/4

(57) ABSTRACT

The invention relates to a tomographic apparatus for the three-dimensional modelling of an object to be imaged, said tomographic apparatus comprising at least one irradiation source for irradiating the object to be modelled from at least two different directions, and at least one receiving means for receiving the radiation transmitted through the object. The reconstruction parameter is a parameter which determines the behavior in iteration of a reconstruction representing the object to be modelled. The tomographic apparatus comprises means for modelling an object to be imaged by using a non-iterative tomographic modelling algorithm for determining reconstruction parameters for a more accurate computation to be conducted with an iterative tomographic modelling algorithm, in which the computation is conducted from a region of interest in the object.

### FIELD OF THE INVENTION

The invention relates to tomographic imaging, particularly to X-ray tomography in medical science.

An object of the invention is to improve the quality of modelling obtained by X-ray tomography, especially over a certain delimited region, or respectively to reduce the time spent for computing an accurate model.

### BRIEF DESCRIPTION OF THE INVENTION

X-ray tomography involves taking several projections through an object. The resulting images are combined by means of a computer for a three-dimensional model. Especially in medical applications, it is important to use a total radiation dose as low as possible. Therefore, it is a general practice to take just a limited number of images, while attempting to focus the radiation solely on a targeted region in an effort to provide a sufficiently good three-dimensional model from a small amount of source data. When using a small number of projection directions for compiling a three-dimensional image, the tomography is referred to as sparse projection data tomography. In particular, if projection images cannot be obtained from everywhere around an object, but instead from a limited opening angle, the tomography is referred to as limited angle tomography.

X-ray tomography is based on the attenuation of a cross-object radiation as it propagates through an object to be examined. The pass-through amount of radiation is measured by means of radiation receiving sensors, for example CCD- or CMOS-technique based matrix sensors or line sensors or traditional film. The radiation passing through an object decays in line with the medium's attenuation capacity. In human body, for example, X-radiation is decayed more by bones than by soft tissues, the shadow of bones being thus visible as a smaller amount of pass-through radiation. The pass-through characteristics of X-radiation are further influenced by its wavelength. The method according to this invention functions also with gamma radiation or other radiation capable of propagating through an object.

In the process of modelling a three-dimensional object, the resulting data is generally arranged in a rectangular coordinate system as imaging elements or voxels. Each triplet of X, Y and Z coordinates indicates a single voxel site to be computed for “a grey tone” indicative of its X-ray transmission, i.e. the attenuation of X-radiation caused by this particular voxel. Attenuations are generally computed by using logarithms of pass-through radiation, because that way the computation only requires the use of linear equations. Attenuations in the propagating direction of a ray beam can be directly summed logarithmically.

A problem is how to produce a three-dimensional image, i.e. a cluster of voxels, from information consisting of two-dimensional images. In certain cases, the same problem can be worked out by way of slices, i.e. ray beams crossing a common plane are computed from at least two two-dimensional images at a time, thus using one-dimensional imaging processes for computing two-dimensional imaging processes, and the final three-dimensional imaging process is effected by integrating the computed planes or sectioned slices for three-dimensional entities. Computation by way of slices is most likely successful in the event that the images taken from various directions have their sources coplanar. Generally speaking, the integration of several images cannot be directly done by way of slices.

It is an object of the invention to provide a more advanced and quicker-than-before method and apparatus for the three-dimensional modelling of an object to be imaged.

This is accomplished by a method for the three-dimensional modelling of an object to be imaged, said method comprising irradiating an object to be modelled from at least two different directions and receiving the radiation transmitted through the object, and the reconstruction parameter in said method being a parameter which determines the behavior in iteration of a reconstruction representing the object to be modelled. In the method, the object is modelled by using a non-iterative tomographic modelling algorithm for determining reconstruction parameters for a more accurate computation to be conducted with an iterative modelling algorithm, said more accurate computation being conducted from a region of interest in the object.

The invention relates further to a method for the three-dimensional modelling of an object to be imaged, said method comprising irradiating an object to be modelled from at least two different directions and receiving the radiation transmitted through the object. In the method, the object is modelled by using a non-iterative tomographic modelling algorithm to provide source values for use in an iterative tomographic modelling algorithm as a first prediction of iteration.

The invention relates also to a method for the three-dimensional modelling of an object to be imaged, the model of said object comprising imaging elements or voxels, and the method comprising irradiating an object to be modelled from at least two different directions and receiving the radiation transmitted through the object. In the method, a non-iterative tomographic modelling algorithm is used for determining a source value for a voxel, which comprises visual information about the voxel, as well as a reconstruction parameter, which comprises information about the probability of the source value being a true value, and said source value and reconstruction parameter are determined for as many voxels as necessary in a computation conducted with an iterative tomographic modelling algorithm, in which use is made of the source value information compiled with the non-iterative tomographic modelling algorithm as well as the reconstruction parameter information, and the computation is conducted from a region of interest in the object.

The invention relates to a tomographic apparatus for the three-dimensional modelling of an object to be imaged, said tomographic apparatus comprising at least one irradiation source for irradiating the object to be modelled from at least two different directions, and at least one receiving means for receiving the radiation transmitted through the object, and the reconstruction parameter is a parameter which determines the behavior in iteration of a reconstruction representing the object to be modelled. The tomographic apparatus comprises means for modelling an object to be imaged by using a non-iterative tomographic modelling algorithm for determining reconstruction parameters for a more accurate computation to be conducted with an iterative tomographic modelling algorithm, in which the calculation is conducted from a region of interest in the object.

The invention relates further to a tomographic apparatus for the three-dimensional modelling of an object to be imaged, said tomographic apparatus comprising at least one irradiation source for irradiating the object to be modelled from at least two different directions, and at least one receiving means for receiving the radiation transmitted through the object. The tomographic apparatus comprises means for creating source values by using a non-iterative tomographic modelling algorithm and using said source values in an iterative tomographic modelling algorithm as a first prediction of iteration.

The invention relates also to a tomographic apparatus for the three-dimensional modelling of an object to be imaged, the model of said object comprising imaging elements or voxels and said tomographic apparatus comprising at least one irradiation source for irradiating the object to be modelled from at least two different directions, and at least one receiving means for receiving the radiation transmitted through the object. The tomographic apparatus comprises means for using a non-iterative tomographic modelling algorithm so as to determine a source value comprising visual information about a voxel, as well as a reconstruction parameter comprising information about the probability of the source value being a true value, and said source value and reconstruction parameter are determined for as many voxels as necessary for an iterative tomographic modelling algorithm, and the tomographic apparatus comprising means for using the iterative tomographic modelling algorithm so as to make use of the source value information compiled with the non-iterative tomographic modelling algorithm, as well as the reconstruction parameter information, and the iterative tomographic modelling algorithm being used for conducting a computation from a region of interest in the object.

The invention is based on having the invention implemented by way of first and second procedures, said procedures being different from each other, and the object to be imaged being divided into imaging elements or voxels. The first, non-iterative procedure is used for determining a source value for a voxel, which comprises visual information about the voxel, and/or a reconstruction parameter, which comprises information about the probability of the source value being a true value. Said source value and reconstruction parameter are determined for as many voxels as necessary in an iterative computation conducted by the second procedure, making use of the source value information compiled by the first procedure, as well as the reconstruction parameter information, and focusing on a region of interest.

The first procedure provides a quick result and the second procedure provides an accurate result. The second computation procedure may take considerably more time than the first one. Said values for a cluster of voxels computed by the first procedure are utilized for conducting a computation in the second procedure. The cluster of voxels produced by the first procedure can be even considerably smaller than a cluster of voxels produced by the second procedure. What is essential is that the second computation procedure in some way utilizes the voxel values computed by the first procedure or the reconstruction parameters related thereto. This utilization can be a first prediction for the second procedure, a delimitation of the space involving a cluster of voxels to define a region of interest prior to computation, the remaining space being computed in a less precise manner. The cluster of voxels provided by the imaging apparatus, computed according to the first procedure, may also function by way of a draft in the actual imaging process and be used in the imaging process for the assessment of imaging success and the actual more accurate image is produced by the second procedure. Thus, a patient can be subjected to new imaging procedures, for example from another direction, prior to a more precise computation. This saves time especially for the patient, as well as for imaging attendants.

The invention offers a benefit of the first procedure producing an image sufficiently accurate for a preliminary examination and an assessment of imaging success, yet being considerably quicker than the second procedure. The first computation procedure is performed quickly, whereby the person responsible for imaging receives a speedy feedback about whether a desired target region is visible in the resulting image at a sufficiently high resolution. Thereafter, the imaging can be focused further, either following a new application of X-radiation, whereby it is thus advantageous to only irradiate the region delimited according to the image produced by the first procedure. It is likewise possible to compute a more accurate image from the previously imaged object, which is as if taken from another direction, and this can be effected without further irradiation. The method according to the invention may even be successful in turning a failed image into a useful, successful one. In addition, when the voxel and reconstruction parameter values computed by the first procedure are used as a first prediction in the iterative computation of the second procedure, the overall speed and accuracy of computation shall be improved.

By virtue of the invention, it will be possible in most cases both to reduce substantially the dosage received by a patient and to speed up the actual imaging process. Since the rapid three-dimensional modelling, computed according to the first procedure, can be performed almost in real time, this enables a confirmation of the directions of interest for further imaging sessions and moreover, prior to a more accurate computation, a reliable message is obtained confirming that the imaging process has been successful. The final three-dimensional image is compiled by means of a slower but more accurate second procedure, preferably by using the results provided by the first procedure as a source value for a computation according to the second procedure.

### LIST OF FIGURES

FIG. 1 shows the way an object to be imaged is divided into imaging elements or voxels,

FIG. 2 shows a process of linear tomography, and

FIG. 3 shows a process of reconstruction computation.

FIG. 4 is a block diagram for an apparatus of the invention.

### DETAILED DESCRIPTION OF THE INVENTION

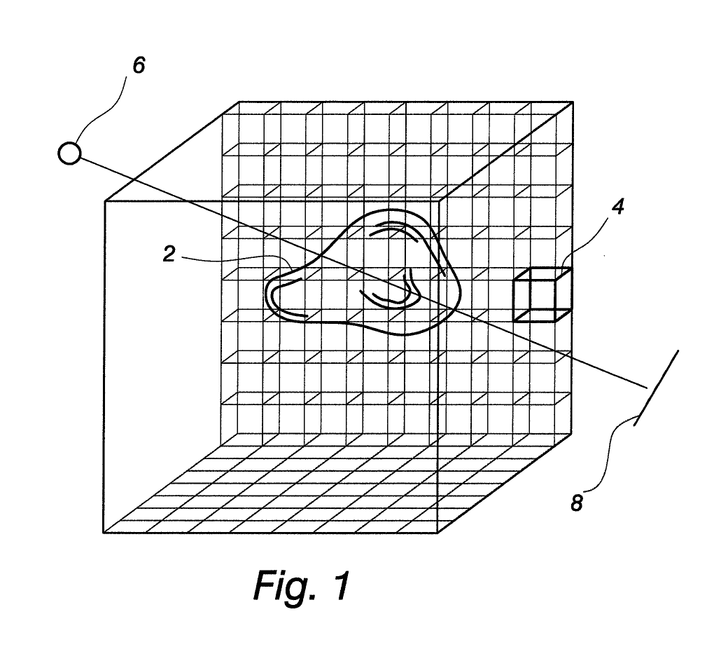

Referring to FIG. 1, there is shown by way of example the fact that, in the process of modelling an object 2 three-dimensionally in medical radiology, the object 2 to be imaged is divided into imaging elements or voxels 4. An X-ray source 6 supplies the object 2 to be imaged with X-radiation. A detector 8 receives the X-radiation transmitted through the object. The rate of X-radiation is lower that that emitted by the X-ray source 6 to the object 2 as some of the X-radiation dies away in the object to be imaged or scatters from the object to be imaged beyond the detector.

The object 2 is modelled mathematically based on the X-radiation received by the detector 8 and the purpose in this modelling is to discover the attenuation of X-radiation in each voxel 4 in order to find out the grey tone of each voxel 4.

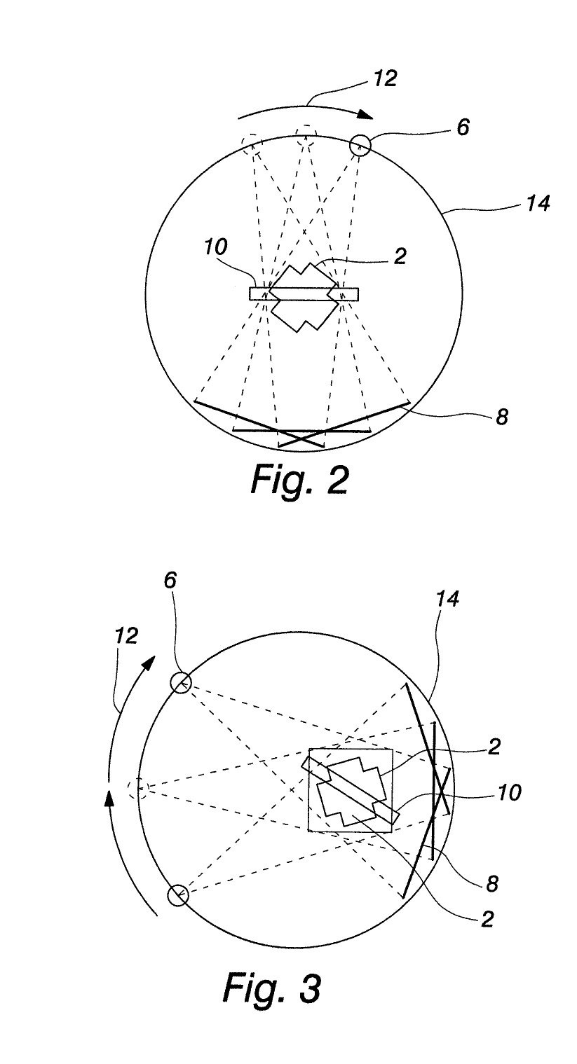

FIG. 2 shows a process of linear tomography. Linear tomography is used for imaging a sharply-defined layer 10 within the object 2. The X-ray source 6 and the detector 8 move along an imaging orbit 14 while imaging by scanning the sharply-defined layer inside the object. The layer 10 has its thickness determined by an angular difference between the extreme ends of a displacement 12. In linear tomography, the formation of a three-dimensional image calls for a high dose of radiation and the imaging process is slow.

FIG. 3 shows a process of reconstruction computation. In reconstruction computation, a three-dimensional modelling process is effected on the object 2 by making use of visual information provided in linear tomographic imaging about the sharply-defined layer 10 and projection images taken for a reconstruction computation from at least two different directions. The reconstruction computation saves generally imaging time and requires a lesser radiation dose than linear tomography.

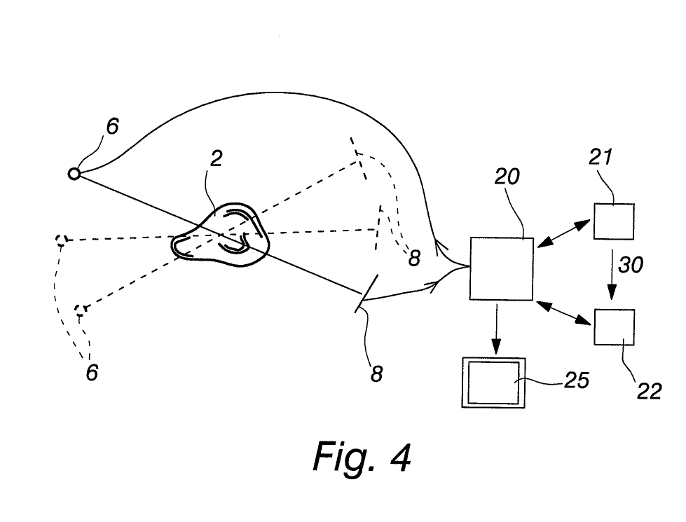

FIG. 4 depicts a block diagram for an imaging apparatus of the invention. A control device 20 may comprise a control computer or comprises apparatus-controlling user interface software which activates an X-ray source 6 and reads the imaging data from a detector or detectors 8. The number of detectors and radiation sources can also be more than one, enabling the imaging process from many directions and enabling also the imaging of a moving object, for example the heart, three-dimensionally. For imaging a stationary object, it is sufficient to use one displaceable radiation source and one preferably displaceable detector. To the control device is linked a display and display interface means 25, whereby the attending staff will be able to examine two-dimensional and computed three-dimensional slice images, as well as to control the imaging process. The control device inputs the detector output data to first reconstruction means 21. On the basis of a first reconstruction computed by the first means, the attending staff can determine the imaging success rate and, if necessary, take additional images for a higher-resolution image of the region of interest. The additional images and reconstruction data 30 computed with the first imaging algorithm are delivered as a source value to second reconstruction procedure means 22, which compute a more accurate reconstruction by using a rough model computed by the first reconstruction means 21 and possibly taken additional images as the source value. In practice, the reconstruction data 30 can also be stored, for example in a disk server, for a subsequent more detailed processing and the reconstruction data produced by a second reconstruction algorithm is stored for a subsequent examination. The imaging apparatus according to the invention provides a high-level certainty that the final reconstruction shall represent the region of interest with a resolution sufficient to render additional images unnecessary. At the same time, every irradiation applied to a patient serves the purpose of imaging as well as possible, nor is any separate exposure necessary for focusing, but all exposures contribute also to a final accurate reconstruction, and all images taken after the first images can be exposed over a delimited region, whereby the unnecessary irradiation of tissues need not be extended beyond the region of interest. Thus, the apparatus of the invention is capable of providing a higher-than-before resolution final reconstruction with a minimal number of irradiations and reducing the number of failed imaging processes. The imaging apparatus is improved also in terms of efficiency, because the region of interest can be defined quickly and reliably and the patient shall spend less time in the imaging process.

The X-ray imaging apparatus of the invention includes means 21, 22 for applying two reconstruction procedures in an imaging process. These means may be common to both computation algorithms, for example one computer software may execute both reconstruction algorithms and provide the reconstruction parameters produced by the first algorithm as parameters for the second algorithm, the algorithms being executed in a single computer processor. The first and second algorithms can also be executed in separate programs simultaneously for separate images or sequentially in a single processor. The means may also be separate computers, said computers being linked for example by means of a local area network to enable a transfer of the parameters and final computation results between the algorithms. This makes it possible that the more stressful second procedure computation be executed in a more powerful second computer and only the first rapid algorithm be executed during the actual imaging process in a computer associated with the X-ray or gamma imaging apparatus itself. This enables the subsequent “development” of higher-resolution images by way of a batch run in a computer or a cluster of computers, which is optimized for iterative computation and which may be common to several pieces of X-ray equipment or may even be common to several hospitals. Distribution of a computation process requires high-capacity communication links, because three-dimensional high resolution data calls for plenty of storage space and transfer capacity. Distributing a computation process to separate computers releases the first algorithm executing computer more quickly after radiography, and the implementation of various stressful iteration methods may be easier to carry out in a distributed mode, in which case the stressful second algorithm executing computer or the cluster of computers executing iteration sequences is not in interactive service with the operator, but instead the use of its storage and computation capacity can only be optimized for stressful iterative computation of a batch run type and only the first algorithm executing computer is in interactive service as far as the operator is concerned. Thus, the second algorithm running computer need not discontinue the computation process in order to vacate storage space for the processing of a next tomographic image, and the storage requirements of the first algorithm running computer are determined by just one storage occupying process and the second algorithm can possibly be executed preferably in several computers simultaneously as in a pipelining manner, the computers outputting an iteration result always to the next computer and accepting a computation result from the preceding computer as input data. The solution is well known in the distribution of iterative computation over several computers.

Reference will now be made to two commonly known algorithms applied in tomography. A TACT algorithm (Tuned-Aperture Computed Tomography®) was developed in Wake Forest University School of Medicine. It enables the formation of a high-speed 3D approximation of an image taken from two or more arbitrary directions. Most of the other methods require a bunch of images from certain directions. TACT (Tuned Aperture Computed Tomography) is a registered trademark of Wake Forest university and has been described along with its applications e.g. in patent publications U.S. Pat. No. 5,359,637, U.S. Pat. No. 5,668,844 and U.S. Pat. No. 6,081,577.

Applications according to the TACT algorithm enable a random choice of imaging directions and a number of images, and thereby a dose. Consequently, the attendants can choose imaging directions and a number of imaging processes with regard to the probability of obtaining an accurate image of the region of interest. The region of interest is referred to as ROI. In the case of all objects, two images are not enough to provide a high quality three-dimensional image. If it is desirable to use several images for computing even more precise modelling processes, it is often an ART algorithm (Algebraic Reconstruction Technique) which is used.

The TACT algorithm is based on straightforward computation, thus producing a quick result. The ART algorithm has its computation based on iterative computation of each voxel value by using Kaczmarz's method for iteration. Since, normally, a single modelling process involves a considerably large number of voxels, the execution of several iterative computation cycles takes a lot of time. However, the ART does produce a more accurate image, provided that a sufficient number of iterations are executed. If the entire information is computed solely by means of an ART algorithm, the results will take an unacceptably long time to come. Furthermore, since it is preferable to use simultaneously taken images without a patient moving in the meantime, it is a good practice to have a region of interest or ROI defined quickly and, if necessary, to take further images for a more precise modelling process.

According to the invention, for example two images are first taken of a region of interest. This is followed by quickly computing a preliminary image with a TACT algorithm or the like non-iterative fast algorithm. At this point, accuracy may be limited in order to obtain the result quickly. Once the first model has been obtained, the attendants will be able to make a decision on that basis whether more images are needed from a certain more restricted region. If that is the case, more images are taken while the patient remains in a substantially unchanged imaging position. This is the easiest way of holding an object to be imaged in the same position and the additional images are taken from a desired region of interest by re-irradiating a minimum amount of tissue. For this purpose, the apparatus is preferably provided with two discrete sets of imaging means (radiation source and detector), the first imaging means being capable of taking two or more images by irradiating an extensive area and the second imaging means being capable of taking additional images by irradiating a more restricted area from one or more directions for a more accurate image. The delimitation can also be effected by means of a single set of imaging means, e.g. by having the radiation passed through an object collimated as appropriate for each imaging sequence. This is followed by having either original or subsequently taken, more limited images processed for a more accurate image by the application of an ART algorithm. Since the algorithm is stressful and iterative, the source used for iteration will be a TACT image for obtaining a focused final result more quickly with less computation. Thus, if further images have indeed been taken, a TACT algorithm will now be used to first compute values for the voxels in a region of interest. Thereafter, an ART algorithm is used for computing a more accurate model regarding the region of interest, and possibly the computation for an area outside the region of interest is facilitated by a three-dimensional image created by the TACT algorithm.

Because the ART algorithm requires a plurality of iteration cycles for gradually focusing the result, the overall computation time can thus be significantly reduced in the process of creating an accurate three-dimensional model. Moreover, with the patient in mind, preliminary results are obtained quickly before high-precision computation, thus avoiding the taking of unnecessary renewed images.

In practice, the ART algorithm refers to forming a group of equations for attenuations caused by voxels that happen to be located along the route of each ray beam that has passed through an object to be imaged and to working out said equations by a method of least square. Hence, providing a reasonably accurate source value as a first prediction speeds up the attainment of a desired iteration accuracy. Since the attenuation of ray beams occurring outside the region of interest undermines the result, it is possible to choose imaging directions which are better than the original ones for an eventually more accurate imaging process. At the same time, an estimate is provided regarding attenuation outside the region of interest since, even though the computation of more accurate values for these particular voxels may not necessary, knowing the same is beneficial in terms of minimizing the shadows external of the region of interest.

The method according to the invention is useful for X-ray tomographic imaging in medicine, for example for breast cancer screening and for a more thorough examination of suspect regions. The method also enables the use of a plurality of X-radiations accelerated with various forms of energy, or gamma radiation as well. It enables taking several images simultaneously as the directions of images are readily selected in such a way that the ray beams do not interfere with each other. Thus, an image is obtained even of a moving object, for example the heart.

Technical procedures more detailed than the one presented above have not been described, because such procedures can be carried out by implementations that are consistent with the prior art in terms of hardware engineering, electronics and software.

Although the invention has been described above in reference to the attached figures and specification, the invention is by no means limited to those but, instead, the invention is highly versatile within the scope allowed by the claims.

1-16. (canceled)

17. A method for the three-dimensional modelling of an object to be imaged, said method comprising irradiating an object to be modelled from at least two different directions and receiving the radiation transmitted through the object, and the reconstruction parameter in said method being a parameter which determines the behavior in iteration of a reconstruction representing the object to be modelled, characterized in that the object is modelled by using a non-iterative tomographic modelling algorithm to provide source values for use in an iterative tomographic modelling algorithm as a first prediction of iteration and the object is modelled by using a non-iterative tomographic modelling algorithm for determining reconstruction parameters for a more accurate computation to be conducted with an iterative modelling algorithm, said more accurate computation being conducted from a region of interest in the object.

18. The method as set forth in claim 17, characterized in that said non-iterative tomographic modelling algorithm is a tomosynthesis algorithm and said iterative tomographic modelling algorithm is an ART algorithm.

19. The method as set forth in claim 17, characterized in that the method comprises the use of X-radiation.

20. The method as set forth in claim 17, characterized in that the method comprises the use of two or more two-dimensional X-ray images taken from arbitrary directions for creating a three-dimensional image.

21. The method as set forth in claim 17, characterized in that the method comprises taking two or more images by irradiating an extensive area and, if necessary, additional images are taken over a more limited region from one or more directions for providing a more accurate image of said more limited region.

22. A method for the three-dimensional modelling of an object to be imaged, the model of said object comprising imaging elements or voxels, and the method comprising irradiating an object to be modelled from at least two different directions and receiving the radiation transmitted through the object, characterized in that a non-iterative tomographic modelling algorithm is used for determining a source value for a voxel, which comprises visual information about the voxel, as well as a reconstruction parameter, which comprises information about the probability of the source value being a true value, and such source value and reconstruction parameter are determined for as many voxels as necessary in a computation conducted with an iterative tomographic modelling algorithm, in which use is made of the source value information compiled with the non-iterative tomographic modelling algorithm as well as the reconstruction parameter information, and the computation is conducted from a region of interest in the object.

23. The method as set forth in claim 22, characterized in that said non-iterative tomographic modelling algorithm is a tomosynthesis algorithm and said iterative tomographic modelling algorithm is an ART algorithm.

24. The method as set forth in claim 22, characterized in that the method comprises the use of X-radiation.

25. The method as set forth in claim 22, characterized in that the method comprises the use of two or more two-dimensional X-ray images taken from arbitrary directions for creating a three-dimensional image.

26. The method as set forth in claim 22, characterized in that the method comprises taking two or more images by irradiating an extensive area and, if necessary, additional images are taken over a more limited region from one or more directions for providing a more accurate image of said more limited region.

27. A tomographic apparatus for the three-dimensional modelling of an object to be imaged, said tomographic apparatus comprising at least one irradiation source for irradiating the object to be modelled from at least two different directions, and at least one receiving means for receiving the radiation transmitted through the object, and the reconstruction parameter is a parameter which determines the behavior in iteration of a reconstruction representing the object to be modelled, characterized in that the tomographic apparatus comprises means for creating source values by using a non-iterative tomographic modelling algorithm and using said source values in an iterative tomographic modelling algorithm as a first prediction of iteration and means for modelling an object to be imaged by using a non-iterative tomographic modelling algorithm for determining reconstruction parameters for a more accurate computation to be conducted with an iterative tomographic modelling algorithm, in which the computation is conducted from a region of interest in the object.

28. The tomographic apparatus as set forth in claim 27, characterized in that said non-iterative tomographic modelling algorithm is a tomosynthesis algorithm and said iterative tomographic modelling algorithm is an ART algorithm.

29. The tomographic apparatus as set forth in claim 27, characterized in that the tomographic apparatus comprises means which are capable, on the basis of data provided by the non-iterative method, of delimiting and changing a region of accurate computation used in the iterative method to confine the accurate computation within a region of interest in the object.

30. The tomographic apparatus as set forth in claim 27, characterized in that the tomographic apparatus comprises means for transposing the three-dimensional data computed by a non-iterative method for the source values of an iterative method for accurate computation.

31. The tomographic apparatus as set forth in claim 27, characterized in that the tomographic apparatus comprises means for using X-radiation in said tomographies.

32. The tomographic apparatus as set forth in claim 27, characterized in that the tomographic apparatus comprises means of using two or more two-dimensional X-ray images, taken from arbitrary directions, for creating a three-dimensional model.

33. The tomographic apparatus as set forth in claim 27, characterized in that the tomographic apparatus comprises imaging means adapted to take at least two images by irradiating an extensive area and necessary additional images by irradiating a more limited region from at least one direction for providing a more accurate image.

34. A tomographic apparatus for the three-dimensional modelling of an object to be imaged, the model of said object comprising imaging elements or voxels and said tomographic apparatus comprising at least one irradiation source for irradiating the object to be modelled from at least two different directions, and at least one receiving means for receiving the radiation transmitted through the object, characterized in that the tomographic apparatus comprises means for using a non-iterative tomographic modelling algorithm so as to determine a source value comprising visual information about a voxel, as well as a reconstruction parameter comprising information about the probability of the source value being a true value and said source value and reconstruction parameter are determined for as many voxels as necessary for an iterative tomographic modelling algorithm, and the tomographic apparatus comprising means for using the iterative tomographic modelling algorithm so as to make use of the source value information compiled with the non-iterative tomographic modelling algorithm, as well as the reconstruction parameter information, and the iterative tomographic modelling algorithm being used for a computation from a region of interest in the object.

35. The tomographic apparatus as set forth in claim 34, characterized in that said non-iterative tomographic modelling algorithm is a tomosynthesis algorithm and said iterative tomographic modelling algorithm is an ART algorithm.

36. The tomographic apparatus as set forth in claim 34, characterized in that the tomographic apparatus comprises means which are capable, on the basis of data provided by the non-iterative method, of delimiting and changing a region of accurate computation used in the iterative method to confine the accurate computation within a region of interest in the object.

37. The tomographic apparatus as set forth in claim 34, characterized in that the tomographic apparatus comprises means for transposing the three-dimensional data computed by a non-iterative method for the source values of an iterative method for accurate computation.

38. The tomographic apparatus as set forth in claim 34, characterized in that the tomographic apparatus comprises means for using X-radiation in said tomographies.

39. The tomographic apparatus as set forth in claim 34, characterized in that the tomographic apparatus comprises means of using two or more two-dimensional X-ray images, taken from arbitrary directions, for creating a three-dimensional model.

40. The tomographic apparatus as set forth in claim 34, characterized in that the tomographic apparatus comprises imaging means adapted to take at least two images by irradiating an extensive area and necessary additional images by irradiating a more limited region from at least one direction for providing a more accurate image.
